(19) United States
(12) Patent Application Publication
(10) Pub. No.: US 2014/0350505 A1
(43) Pub. Date: Nov. 27, 2014

(54) Keneric brief pouch

(71) Applicant: Kenneth Mack Williams, San Jose, CA (US)

(72) Inventor: Kenneth Mack Williams, San Jose, CA (US)

(21) Appl. No.: 14/120,558

(22) Filed: Feb. 28, 2011

(51) Int. Cl. A61F 13/56; A61F 13/474; A61F 13/471

(52) U.S. Cl. CPC A61F 13/5611; A61F 13/471; A61F 13/474
USPC 604/385.03

(57) ABSTRACT

The Keneric brief pouch is a thin miniature absorbent pouch that attaches to Under garments. It is designed to capture leaking body fluids after urination In a secured pouch. It is attached with an adhesive backing to under Garments to hold the male organ in place to prevent staining to clothing after Urination. Keneric brief pouches can be discarded after use or being worn t To prevent incontinence accidents.

### FIELD OF INVENTION

The present invention relates to disposable adhesive underwear apparatus for Men's briefs.

### BACKGROUND OF INVENTION

Keneric Brief Pads are a special garment to be worn in side of underwear. Absorbent material garment pads, clothing adhered pads.

Herein describes small cloth ad hearable pouches which attach to the inside Of men's briefs, such as boxers, pajama's pants etc, with an adhesive skin That attaches inside briefs facing inside. This absorbent pouch is to mute the The possibility of moisture emitted by urination, thus preventing any Unwanted moisture or stains to clothing. The pouch is easily removed when It is no longer needed. It is designed primarily for use in treating male Incontinence. It is described as a composite undergarment of multiplayer Construction using a contact layer of adhesive material with super absorbent And high moisture transmission layers attached.

The invention in this group relate to garments or undergarments. Clothing Attached brief pouches that are used as an addition to the inside of the briefs

### SUMMARY OF THE INVENTION

In keeping with these objects and others which may become apparent, The Present invention is a Keneric Brief Pouch that is invisible from the exterior And which can be used with any form fitting attire with the use of under Pants or any undergarment.

Since the Keneric Brief Pouch is adherably only to the upper crotch facing Area of an outer pant garment, It is unencumbered by auxiliary support, such As annoying skin adhering adhesives or other body supports, such as waist Bands or belts.

Constructed of a thin absorbent pouch, the Keneric brief pouch of present Invention is adhesively bonded temporarily during use to the crotch area of The inner garment. It can be used in jeans, exercise pants, thermal Underwear, shorts, or any other encompassing garment worn. The Keneric Brief Pouch is comfortable and helps prevents infection from unabsorbed Urine. This invention provides a clean bacteria-free pouch. It absorbs Moisture and also provides extra protection for garments.

### BRIEF DESCRIPTION OF THE DRAWINGS

The present invention can best be under stood in connection with the Accompanying drawings. It is noted that the invention is not limited To the precise embodiments shown in the drawing

FIG. 1 is a perspective top front view of a Keneric Brief Pouch of this Invention;

FIG. 2 is a straight front view of a Keneric Brief Pouch of this invention;

FIG. 3 is a side view of a Keneric Brief Pouch of this invention;

FIG. 4 is aback view of a Keneric Brief pouch of this invention;

FIG. 5 is a perspective view inside Keneric Brief Pouch of this invention;

FIG. 6 is a level front view of the bottom of a Keneric Brief Pouch of this Invention;

### DETAILED DESCRIPTION OF THE INVENTION

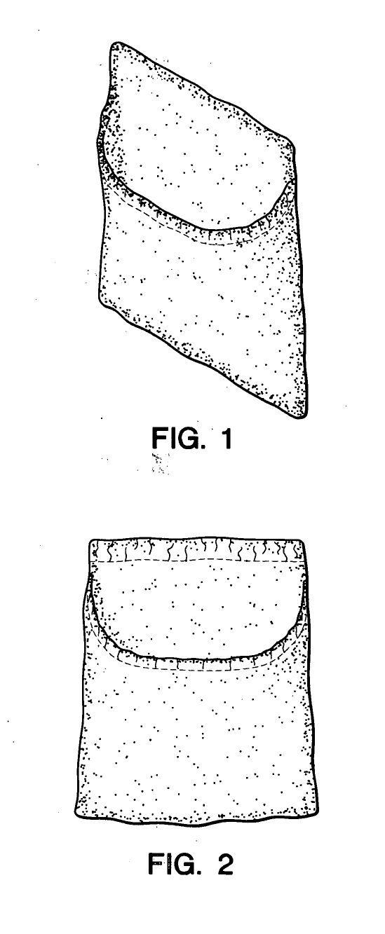

FIG. 1 the user derives sanitary protection from un-wanted urine residue. Keneric Brief Pouch traps moisture in a sanitary napkin pouch that is Covered with a thin outer layer of polyurethane to prevent leaking. The Keneric Brief Pouch of this invention are intended to be worn in the crotch Area.

FIG. 2 shows the Keneric Brief Pouch pad front view with bonded edges And bottom area. The front ridge area is an elastic barrier that pulls open. The elastic ridge makes for a snug fit to the wearer.

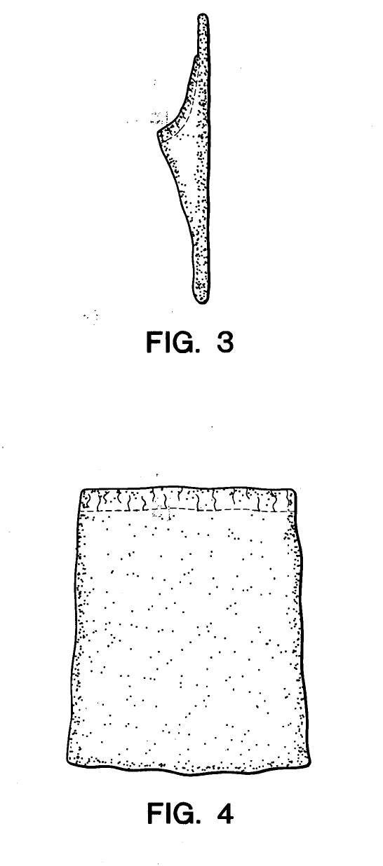

FIG. 3 shows the construction of alternate embodiment of the Keneric Brief Pouch of this invention. It includes at least three surfaces bonded layers with Adhesive patches on the bottom layer. Top layer is a thin permeable non Woven fabric material, such as of a paper or a synthetic plastic material. However. Top layer may also be woven material. Middle layer is a thin Absorbent non-woven or woven material, and the bottom layer is a thin Impermeable layer, such as of a synthetic plastic. Total thickness of the Keneric Brief Pouch must be kept to a minimum to prevent a visible Outline from showing through.

FIG. 4 shows the back of the Keneric Brief Pouch. A thin adhesive strip Is attached to this portion of the Keneric Brief Pouch. The strip can be peeled, then the Keneric Brief Pouch can be applied and worn in the inside crotch area of underwear.

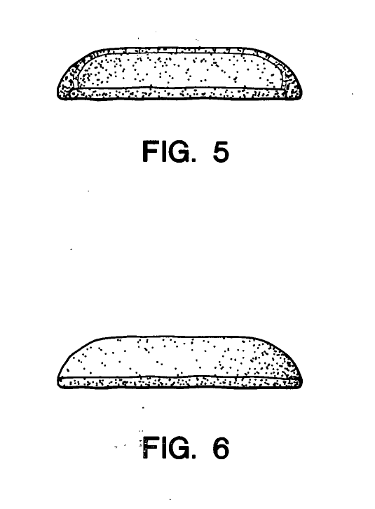

FIG. 5 shows the inside pocket area of the Keneric Brief Pouch with the Circumference around the Keneric Brief Pouch.

FIG. 6 shows the bottom area of the Keneric Brief Pouch and thickness Of pad.

What is claimed is:

1. A post-on disposable Keneric Brief Pouch comprising:
A body having a permeable top sheet, a liquid impermeable back sheet, and
An absorbent member interposed between the top sheet and back sheet;
A stomach portion located on a wearer's crotch side respectively, of said Crotch portion to form a unitary opening portion;

2. The adhesive stick-on disposable Keneric brief pouch as claimed in claim 1, wherein said is all formed by absorbent fibers and elastic Members.

3. The adhesive stick-on disposable Keneric brief pouch is claimed in claim 1, wherein adhesive strip f the under garment are substantially Attached.

4. The adhesive stick-on disposable Keneric brief pouch comprising;
A body having a liquid permeable top sheet, a liquid impermeable back Sheet, and an absorbent member interposed between the top sheet and Back sheet;

5. The adhesive stick-on disposable Keneric brief pouch as claimed in claim 4, wherein said gathers are all formed by elastic members;

6. the adhesive stick-on disposable Keneric brief pouch.
